(19) United States
(12) Patent Application Publication
(10) Pub. No.: US 2022/0133518 A1
(43) Pub. Date: May 5, 2022

(54) SPECIALIZED ORTHOTIC FOOT BRACE WITH BOTH BILATERAL AND UNILATERAL SUPPORT AND AUTO LIFT FUNCTIONS

(71) Applicant: Gregory Jon Coggins, Landrum, SC (US)

(72) Inventor: Gregory Jon Coggins, Landrum, SC (US)

(21) Appl. No.: 16/949,595

(22) Filed: Nov. 5, 2020

(51) Int. Cl. A61F 5/01

(52) U.S. Cl. CPC A61F 5/0127; A61F 2005/0137

(57) ABSTRACT

An orthotic foot brace provides both bilateral and unilateral support combined with auto lift functionality. The orthotic device has a foot plate and a leg brace operatively associated with a rack and pinion combination, wherein the rotation of the pinion powered through flexion of the ankle is converted to linear motion of the rack and vice versa.

### BACKGROUND OF THE INVENTION

The present invention relates to an orthotic device and more particularly, an orthotic foot brace with both bilateral and unilateral support and auto lift functions.

Foot drop is an abnormal situation in which the dropping of the foot happens due to weakness, irritation, nerve damage, or paralysis of the muscles in the anterior portion of the lower leg.

Current orthotic devices to assist people with foot drop utilize springs or are fixed position devices, both limit motion and prevent the freedom of natural movement and flexion.

As can be seen, there is a need for a device that helps to reduce trips and falls due to foot drop while allowing the wearer to have more natural movement and stability through unilateral and bilateral support in addition to foot-lifting assistance.

The present invention embodies a gas charged shock drive rack and pinion system plus a swinging foot plate adapted to allow freedom, natural movement and flexion of the foot and ankle.

By using a unique shock system with inherent unilateral side support and a pinion type of motion to lift the foot, a natural swinging of the foot is produced during normal heel to toe walking. Additionally, when the present invention is assembled, the present invention provides the bilateral support of the lower extremities for safer, more natural movement of the foot and ankle.

### SUMMARY OF THE INVENTION

In one aspect of the present invention, an orthotic foot brace provides both bilateral and unilateral support combined with auto lift functionality through an orthotic device providing the following: a foot plate; a brace; and one or more rack and pinion combinations operatively associating the brace to a heel portion of the foot plate.

In another aspect of the present invention, the orthotic device may also include wherein said heel portion is dimensioned and adapted to accommodate a heel of a human user; a heel wall extending from a periphery of the heel portion, wherein the pinion of each rack and pinion combination is attached to the heel wall; a shock attached to the brace, the shock having a compensation chamber that slidably receives a piston portion of the rack of the rack and pinion combination, wherein the brace is at least one rigid body fixed relative to a lower leg of the human user, wherein the brace further comprises one or more straps that engage said lower leg.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention, illustrating a flat position;

FIG. 2 is a perspective view of an exemplary embodiment of the present invention, illustrating a lifted position;

FIG. 3 is a side elevation view of an exemplary embodiment of the present invention, shown in use illustrating the flat position;

FIG. 4 is a side elevation view of an exemplary embodiment of the present invention, shown in use illustrating the lifted position;

FIG. 5 is an exploded perspective view of an exemplary embodiment of the present invention, illustrating a shock and gear setup;

FIG. 6 is a section view of an exemplary embodiment of the present invention, taken along line 6-6 of FIG. 5;

FIG. 7 is a perspective view of an exemplary embodiment of the present invention, illustrating the lifted position;

FIG. 8 is a perspective view of an exemplary embodiment of the present invention, illustrating the flat position; and

FIG. 9 is a perspective view an exemplary embodiment of the present invention, illustrating the flat position.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, the present invention designs provide an orthotic foot brace giving bilateral and unilateral support to the leg and ankle combined with auto lift function for the toe area of the foot.

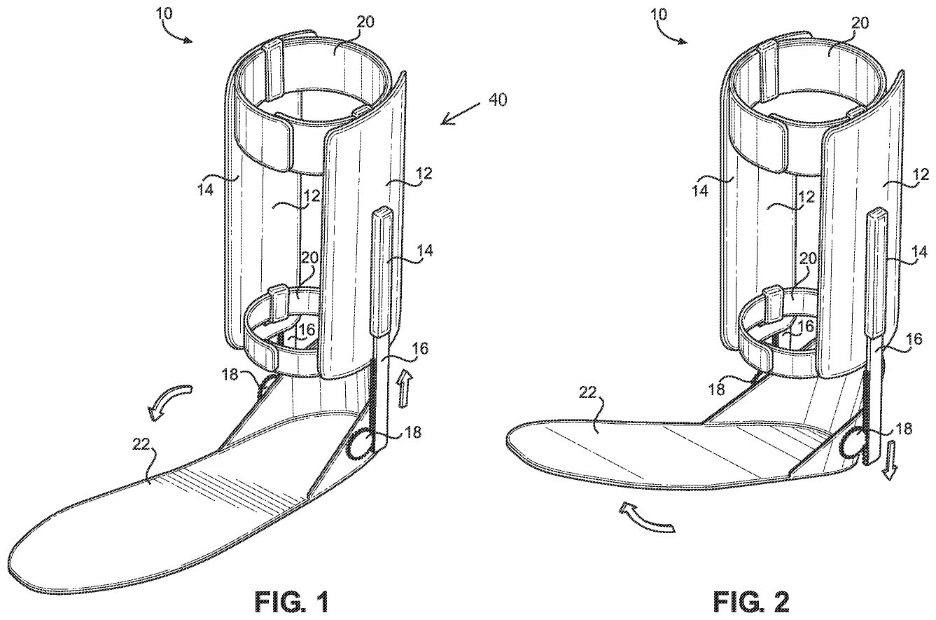

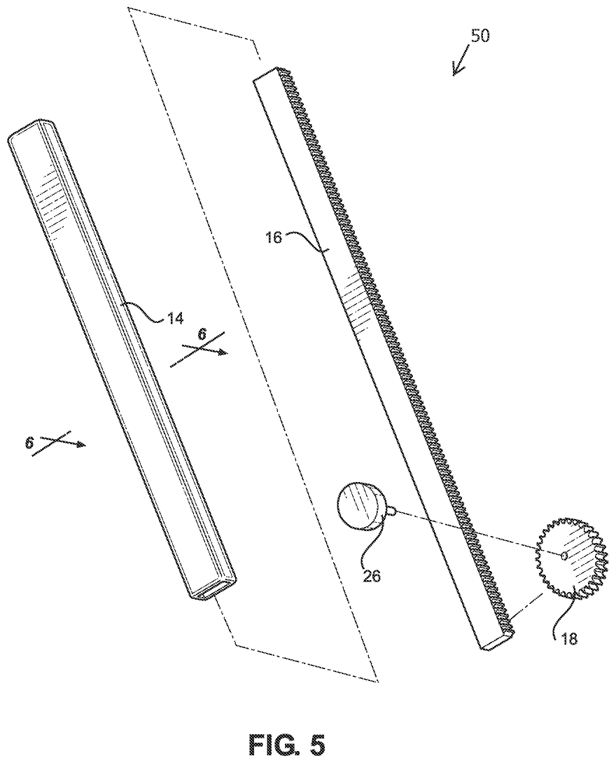

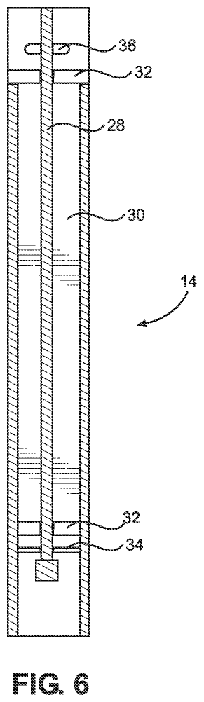

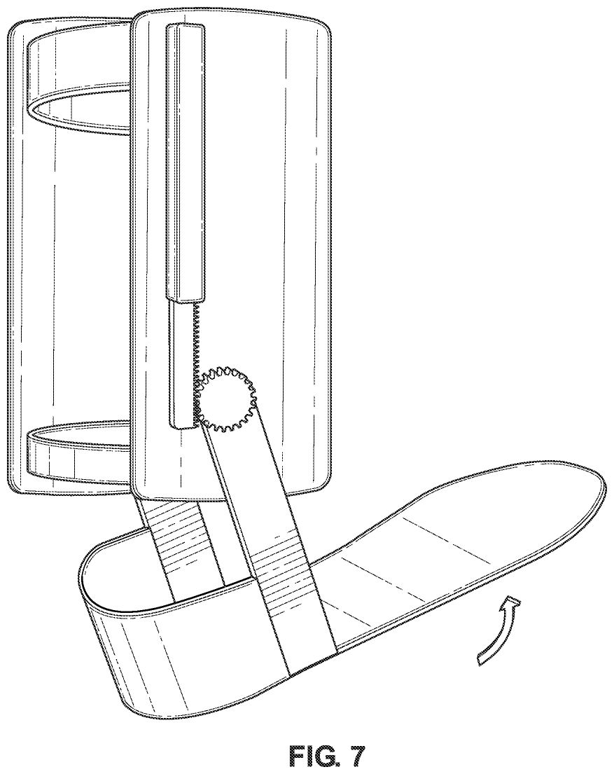

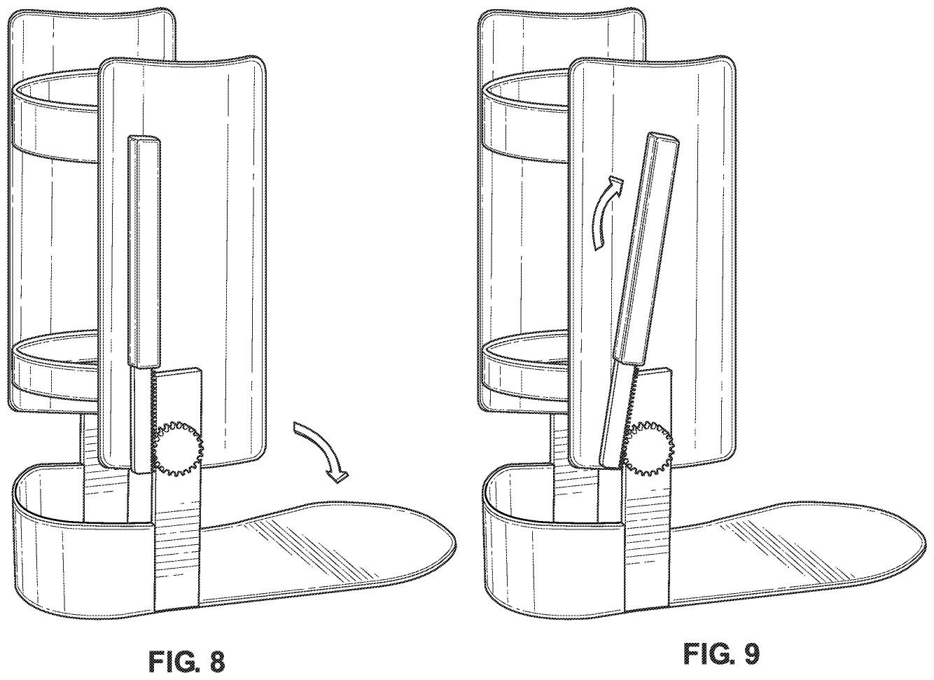

Referring now to FIGS. 1 through 9, the present invention may include an orthotic device 10. The orthotic device 10 has a foot plate 22 and a leg brace 40 operatively associated with a rack and pinion combination 50, wherein the rotation of the pinion 18 powered through flexion of the ankle is converted to linear motion of the rack 16 and vice versa.

The leg brace 40 may include two opposing rigid bodies 12 (or two opposing rigid surfaces of the same body) on each side a lower leg of a human wearer 24, wherein the rigid bodies/surfaces 12 are fixed relative to said lower leg of a human wearer 24. In certain embodiments, straps 20 keep the rigid bodies 12 fixed relative to the lower leg of a human wearer 24.

A shock 14 will be fixed to the exterior of each body 12. Slidably received in a compensation chamber 30 defined by the shock 14 is piston portion of the rack 16, wherein the piston portion slides when urged by the rotation of the pinion 18. The piston portion may or may not have rack teeth. The shock 14 may provide a rod 28 within the compensation chamber 30 along with one or more rod guides 32, a seal 34 and a piston rod seal 36, wherein in some embodiments, the pinion 18 may be eliminated as the direct connection of the piston portion to the hinged foot plate 22 or the heel cup still allows for a smooth and fluid motion. In these embodiments, the piston portion may have a circular shape at the end with a hold for a direct screw mounting or for a roll pin mount.

In some embodiments, the pinion 18 may protrude from vertical heel walls, where the heel walls extend from a rear portion of the foot plate 22 forming a ‘heel cup’. A pinion mount 26 may rotatably connect the pinion 18 to said vertical heel walls. By directly or indirectly spanning between the leg brace 40 and the foot plate 22, the racks 14 on each side of the ankle of the user provide lateral support for the ankle.

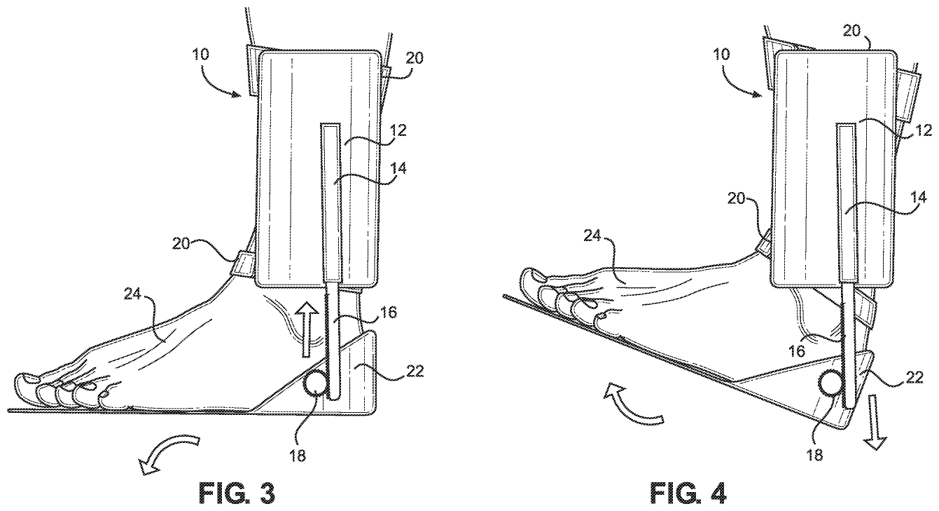

The present invention is designed so that when a user puts weight on a heel portion of the foot plate 22 that is on a supporting surface, as illustrated in FIG. 3, the rack 16/piston portion is urged upward into the shock's compensation chamber 30, which in turn causes the pinion 18 to rotate counterclockwise along the teeth of the rack 16, as illustrated in FIG. 3—forcing the forefoot portion of the foot plate 22 to likewise rotate into the flat position.

Referring the FIG. 4, as a step is taken, the gas charged shock 14 drives the piston portion of the rack 16 downward with more than a eighty pounds of pressure (160 lbs. tot al per device as there are two gas charged shocks per orthotic leg brace) which in turn causes clockwise rotation of the pinion 18 at or adjacent the heel portion of the foot plate 22, thereby rotating the forefoot portion of the foot plate 22 upward. For a wearer suffering from drop foot, their forefoot would otherwise be dropped downward. In certain embodiments, this upwarp rotation causes the forefoot to rise approximately one inch, allowing for the leg to be moved forward in a natural step motion and not catch the toe causing a trip or stumble, reducing trips and falls as the wearer takes steps over sometimes uneven supporting surfaces. Then as the step reaches its completion the heel is placed back on the supporting and the forefoot presses downward causing the piston portion to be driven back up into the shock 14, as illustrated in FIG. 3. The foot plate 22 may rotate about a pivot point defined by the pinion 18 (possibly provided by the pinion mount 26).

A method of using the present invention may include the following. The orthotic device 10 disclosed above may be provided. A wearer may lace the foot into the foot plate and heal cup like a shoe and then tighten the straps across the upper part of the lower leg and just above the ankle. Then the user may slide their foot into the shoe of choice and walk as normal as the present invention operates as explained above.

Additionally, the shock can be separated out and used in almost any industry or setting that currently uses or might need a shock. Because of the slim line and flat design, the inventive concept of the shock can go places typical round shocks do not fit properly. Thus, allowing any design to be tightened up and look sleeker.

It should be understood, of course, that the foregoing relates to exemplary embodiments of the invention and that modifications may be made without departing from the spirit and scope of the invention as set forth in the following claims.

What is claimed is:

1. An orthotic device comprising:
a foot plate;
a brace; two gas charged shocks per brace/Orthotic and
one or more rack and pinion combinations operatively associating the brace to a heel portion of the foot plate.

2. The orthotic device of claim 1, wherein said heel portion is dimensioned and adapted to accommodate a heel of a human user.

3. The orthotic device of claim 2, further comprising a heel wall extending from a periphery of the heel portion.

4. The orthotic device of claim 3, wherein the pinion of each rack and pinion combination is attached to the heel wall.

5. The orthotic device of claim 4, further comprising a shock attached to the brace, the shock having a compensation chamber that slidably receives a piston portion of the rack of the rack and pinion combination.

6. The orthotic device of claim 5, wherein the brace is at least one rigid body fixed relative to a lower leg of the human user.

7. The orthotic device of claim 6, wherein the brace further comprises one or more straps that engage said lower leg.
